(19) United States
(12) Patent Application Publication
(10) Pub. No.: US 2015/0147792 A1
(43) Pub. Date: May 28, 2015

(54) Betaine Enhancement of Fermentation to make C4 Diacids

(71) Applicant: ARCHER DANIELS MIDLAND COMPANY, Decatur, IL (US)

(72) Inventor: Jill Morriss, Blue Mound, IL (US)

(73) Assignee: Archer Daniels Midland Company

(21) Appl. No.: 14/402,347

(22) PCT Filed: Jun. 27, 2013
(86) PCT No.: PCT/US2013/048032

§ 371 Date: Nov. 20, 2014

Related U.S. Application Data
(60) Provisional application No. 61/666,172, filed on Jun. 29, 2012.

(51) Int. Cl. C12P 7/46; C12N 1/20

(52) U.S. Cl. CPC C12P 7/46; C12N 1/20
USPC 435/145; 435/244; 435/252.1

(57) ABSTRACT

A method of making a C4 diacid from a sugar source by fermentation with a microorganism is described that includes supplementing the fermentation medium with relatively low amounts of betaine. The use of betaine can substantially reduce or even completely eliminate the need for complex nutrient source such as yeast extract, and eliminate a need for vitamins such as biotin, while maintaining a high level of production of the C4 diacid from a simplified sugar based media. The use of betaine improves yield from sugar, final titer, and rate of production per hour. The method is particularly suitable for microorganisms that otherwise display a dependence on yeast extract for high productivity, more particularly for any bacteria, and most particularly for the production of succinic acid from the bacterium Actinobacillus succinogenes. An improved strain of A succinogenes is also described, having a mutation in a betaine transporter that improves C4 diacid production in the presence of betaine.

### CROSS REFERENCE TO RELATED APPLICATIONS

This Application claims priority to U.S. Provisional Application No. 61/656,172 filed Jun. 29, 2012.

### BACKGROUND OF THE INVENTION

In laboratory research on developing microorganisms for the purpose of fermentation processes often the initial wild type organism selected for development is propagated in rich fermentation media, such as Luria Broth, which contains yeast extract as a vitamin and nitrogen rich nutrient source. Yeast extract is, however, a relatively expensive nutrient source making it uneconomical to use for large scale industrial fermentation. Therefore, producers of products made by fermentation typically seek to select or adapt an organism for growth on a less expensive nutrient rich media such as for example a molasses, corn steep liquor or a raffinate.

A molasses is a crude carbon rich byproduct stream obtained from refining a major product from a plant material, for example an aqueous side stream obtained from refining sugar from sugar cane or refining oil from soybeans. Corn steep liquor (CSL) is the byproduct water remaining after steeping corn grain in water in a corn wet milling operation. Raffinate is the residual material remaining after refining an amino acid or other product made by a fermentation process. These materials are much less expensive than yeast extract, however, not all organisms can maintain the level of productivity demonstrated from growth on yeast extract when attempts are made to adapt them for growth on a less expensive nutrient source. One such organism exhibiting this problem is Actinobacillus succinogenes, such as described in U.S. Pat. Nos. 5,504,004, 5,723,322, 5,5573,931 and 8,119,377 which may be used to produce succinic acid as a product of glucose fermentation, but requires yeast extract to obtain high productivity rates.

The production of succinic acid through fermentation is a widely published topic. Researchers have attempted to replace the commercial yeast extract with less expensive, nutritive protein sources such as whey protein, soy protein, CSL, stillage spent brewers yeast, etc. In all of the known publications, yield, titer and/or rate of the fermentation significantly declines when the commercial yeast extract is omitted. Vitamin and amino acid supplementation also has been attempted with little success.

For example, Economical Succinic Acid Production From Cone Molasses By Actinobacillus Succinogenes, Yu-Peng Liu, Pu Zheng, Zhi-Hao Sun, Ye Ni, Jin-dun Dong, Lie-Lei Zhu describes a fed batch fermentation conducted on cane molasses Oils varying amounts of yeast extract producing a titer of 55.2 g/l succinic add at a productivity of 1.15 g/l/hr, which is too low to be economical. Substrate inhibition was speculated to be a problem.

In Succinic Acid Production by Actinobacillus succinogenes using spent Brewer's Yeast Hydrolysate as a Nitrogen Source”, Min Jiang, Keiquan Chen, Zhongmin Liu, Ping Wei, et, Al. the authors stated that a nutrient limitation existed when hydrolyzed brewer's yeast was used to replace commercial yeast extract in the media formulation, and that vitamins were the limiting factor. The authors at best obtained 68.8% yield from the sugar source at a productivity rate of less than 1.4 g/h/hr.

In Enhanced Production Of Succinic Acid By Actinobacillus Succinogenes With Reductive Carbon Source, Jian Li, Min Jiang, Kequan Chen, Longan Shang, Ping Wei, Hanjie Ying, Qi Ye, Pingkai Ouyang, Honam Chang described a process where sorbitol was used as a possible carbon substrate for maximizing the redox potential of the succinic add fermentation. Here again the rate was too low and 15 g/l yeast extract was still used as part of the medium.

In Fermentative production of succinic acid from straw hydrolysate by Actinobacillus succinogenes Xiaojiang Fang, Jian Li, Xiaoyu Zheng, Yonglan Xi, Kequan Chen, Ping Wei, Ping-Kai Ouyang, Min Jiang the authors added an osmoprotectant (proline) to their media which enhanced the growth of A. succinogenes giving a 22% increase in productivity, however, the productivity increase was still not sufficient to provide an economically viable alternative for the required yeast extraction.

There is, therefore, a general need in the art to discover an economically viable alternative to using yeast extract for producing C4 diacids via fermentation by microorganisms that snow high productivity with a requirement for the yeast extract. In a particular case, there is a need to eliminate yeast extract as a requirement for economical production of succinic acid from Actinobocillus succinogenes.

### SUMMARY OF THE INVENTION

Disclosed herein is the discovery that betaine can be used to greatly increase the yield, titer and productivity of microorganisms used to make C4 diacids and reduce or eliminate a requirement for yeast extract when a microorganisms is grown on inexpensive nutrient sources. A C4 diacid is defined herein as at least one of succinic, fumaric, malic, maleic and tartaric acid. The disclosure is exemplified by demonstrating substantial improvement in succinic add productivity when Actinobacillus succinogenes is grown on corn steep media as the primary nutrient source, supplemented with small amounts (0.01-1 g/liter) of betaine, even in the absence of yeast extract. Although the disclosure is exemplified with A. succinogenes, the findings with respect to betaine are applicable to other species of microorganisms used to produce C4 diacids including without limitation, other bacteria and the fungi Asperigillis oryzae and Rhizopus oryzae.

Also described is the development of a novel media for the production of succinic add through fermentation, the media being comprised of corn steep liquor as the primary nutrient source, dextrose or other sugar as the primary carbon source, and betaine present in amounts from 0.1 to 1.0 g/liter. The lowest concentration of betaine tested was 0.125 g/liter, which in certain instances provided the highest titers and yields relative to the highest amount tested of 1.0 g/liter. Hence the most effective range of betaine is likely to be between 0.01 and 1.0 gram liter. Furthermore, when an effective amount of betaine is provided in a fermentation media, vitamins, including biotin, can be eliminated without statistically affecting yields or titer.

Further described in a novel derivative strain of A. succinogenes herein designated ME2.7F that was obtained by successive adaptation of fast growing colonies of parent strain FZ45 830.00 by successive passage on media containing corn steep liquor as the primary nutrient source and glucose as the sole added carbon source, the strain being adapted for growth on such a media in the absence of yeast extract but in the presence of betaine.

The addition of betaine free base (anhydrous) to a fermentation media allowed for the elimination of commercial yeast extract without a decrease in productivity. The fermentation costs of succinic acid per pound of finished product produced with commercial yeast extract was 5× more than that of the reformulated, yeast extract-free, media supplemented with betaine.

A new media formulation was also developed using a strain closely related to A. succinogenes FZ45830.60, in which the need for the yeast homogenate or yeast extract was eliminated. In addition, biotin zooid be eliminated from the media without affecting productivity, titer or yield.

### DETAILED DESCRIPTION OF THE INVENTION

Disclosed herein is an improved method of fermenting a microorganism to produce a C4 diacid, which improvement utilizes a betaine in the fermentation medium to eliminate or reduce the need for expensive media components such as yeast extract, and which can more than double the titer, yield and productivity rate from such less expensive media in comparison to the same media lacking the betaine. The need for using yeast extract was abolished and could be substituted with corn steep liquor and betaine and optionally with mono sodium glutamate, while achieving similar or better C4 diacid production parameters as were previously obtained in media requiring yeast extract. The savings in material reduces the overall cost of (A diacid production by 5 fold in comparison to use of yeast extract containing media.

A betaine is a zwitterionic quaternary amine compound having the general structure:

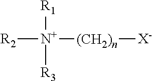

where R₁, R₂ and R₃ are typically small aliphatic groups that are usually the same but which can be different and X is an ionizable anionic functional group, such as carboxylate, sulfate, phosphate, etc. Such compounds exist in zwiterionic form over a wide range of pH values. The most well-known and commercially available betaine is N,N,N-trimethylglycine, originally obtained from beets, where each of R₁, R₂ and R₃ is methyl, n is 1, and X⁻ is carboxylate also known as glycine betaine to distinguish it from other betaines that are widely distributed in microorganisms, plants and animals. Where the present disclosure makes exemplary reference to “betaine” the species was the trimethylglycine, however, the invention may be embodied with Other types of betaines.

The present discovery is pertinent to “C4 diacid producing microorganisms”. All organisms that utilize tricarboxylic acid cycle necessarily pr educe some succinic acid, fumaric acid and malic acid as matter of ordinary metabolism. Some strains of microorganisms can, however, produce C4 diacids to a level much higher than needed for ordinary metabolic growth. Therefore, to distinguish high C4 diacid producing organisms from other organisms, the term “C4 diacid producing microorganism” is defined herein as a microorganism that can ferment a sugar source to make a C4 diacid as a product so that at an endpoint of the fermentation (defined by the time when the sugar is exhausted from the fermentation medium or consumption of the sugar ceases) the fermentation will achieve a titer of at least 20 g/l of the C4 diacid with a conversion yield of at least 10% (defined by wt/wt ratio of the C4 diacid produced to glucose consumed). This definition applies to any fermentation medium where a sugar is the primary carbon source. Examples of C4 producing microorganisms that meet this definition include, without limitation, the bacterium Actinobacillus. succinogenes, which over produces succinic acid and various strains of the fungi Rhyzopus oryzae and Aspergillus oryzae, which can over produce fumaric and malic acid, respectively.

The first exemplary demonstration of the beneficial effects of betaine on C4 diacid production utilized a strain of A. succinogenes designated FZ45 830.60 that was obtained from Michigan Biotechnology institute (a.k.a., MBI, Lansing, Mich.) and has characteristics described in U.S. Pat. No. 8,119,377, and which is on deposit, or was derived from a strain on deposit at the American Type Culture Collection (ATCC) under ATCC Accession Number PTA-6255. In a medium containing 90-155 g/l of dextrose as the primary carbon source supplemented with 15 g/liter of a commercial yeast extract (TASTONE™ 900AG from Sensient, Milwaukee, Wis.) the 830.60 strain was capable of producing a titer of 100 g/l of succinic acid with a 85-90% yield from dextrose at a productivity rate of 2 To 3 g/liter/hour.

For the present work, a variety of media alterations were tried in an attempt to substitute the commercial yeast extract with corn steep liquor and/or with a locally produced homogenized yeast extract made by homogenizing aerobically grown yeast produced incident to ethanol fermentation, and/or by supplementing the media with betaine and/or glutamate. To distinguish commercial yeast extract, which is available from distributors a dry powder, from a homogenate of yeast cells made locally by rupturing yeast cells in liquid suspension, the latter is referred to herein as “local yeast homogenate”

The results of several experiments are shown in Table 1. Corn steep liquor (CSL) was used as the target nutrient source to substitute for yeast extract. CSL is a by-product of wet corn milling obtained by steeping corn grain in water containing a small amount of a sulfide compound for several hours. CSL is rich in low molecular weight organic acids, some carbohydrates, vitamins and minerals that are released from the corn grain. The amount of CSL and local yeast homogenate is shown in terms of dry solids basis (dsb) meaning the amount of material added excluding water weight, per liter of fermentation. Glutamate was added in the form of monosodium glutamate (MSG) and betaine was added in the form of the anhydrous base in the amounts shown. Dextrose was present at between 90 and 125 g/l in all starting media.

The experiments summarized in row 1 of Table 1, show the 860.30 strain is less productive on CSL alone than in the original media used for its propagation that contained similar components and amounts but included the commercial yeast extract at 15 g/l. The highest titer obtained after a prolonged fermentation period of 73.5 hours in the absence of yeast extract or local yeast homogenate was only 28.5 g/liter, at which time only 20% of the glucose was converted to succinate and glucose consumption ceased, leaving behind a high amount of residual glucose. Reducing the amount of CSL to half the amount and adding 15 g/l of local yeast homogenate and 1.3 g/l of MSG as a replacement of the commercial yeast extract improved the fermentation by a factor of about 1.5 to give a titer of 41.2 g/l and a yield from glucose of 30% after only 45 hours at which time glucose consumption ceased as shown in the experiments summarized in row 4.

Strikingly, however, in duplicate experiments summarized in rows 3-5 where betaine was added at only 1 g/liter, the titer obtained was 90-104 g/liter and the yield from glucose soared to about 85%, each value more than double that obtained with the local yeast homogenate in the absence of betaine and more than quadruple that obtained in the absence of any source of yeast derived material. Moreover the fermentation proceeded more quickly, terminating with near exhaustion of the glucose after about 38 hours, corresponding to a productivity rate of 2.4-2.9 g per liter per hour—more than six times the rate obtained in the absence of yeast extract and betaine, and more than twice the rate obtained in the presence of local yeast homogenate in the absence of betaine.

The amount of the mono sodium glutamate did not substantially affect the enhancing effect of betaine. The results shown in rows 6-8 indicate that when MSG was reduced to 0.2 g/l, one sixth the amount used in experiments 2-5, the titer, yield and productivity rate of the culture remained as high, or even higher than obtained with the higher amount of MSG, even when the betaine was reduced to only 0.5 g/l, which in fact gave the highest productivity rate with the MSG also reduced to 0.2 g/l.

In another set of experiments shown in rows 9-11, it was again demonstrated that betaine is responsible for lion's share of the increase in succinic add production and that even low amounts work as well or better than high amounts. When betaine was present in the media at only 0.123 g/l the titer, yield and productivity were the greatest, better than the results obtained with double the amount of betaine, and nearly twice that obtained in the absence of betaine. It is believed that amounts as low as 0.01 g/l.

To determine if betaine would improve the ability of the strain to produce succinate acid on media with a reduced amount of local yeast homogenate and an increased amount of CSL, betaine was tested at various amounts in the presence of 20 g/l CSL solids and only 5 g/l of the local yeast homogenate. The results in rows 12-15 confirmed again that betaine at the lowest tested amount of 0.125 g/l provided the best production in terms of titer, yield and productivity rate and that the overall productivity was nearly two fold that obtained in the absence of betaine. Moreover, the overall productivity in the presence of CSL was nearly as good to even better than obtained with 15 g/liter of the local yeast homogenate. Although the ability to use CSL with reduced amounts of local yeast homogenate was clear from these results, the complete absence of any yeast homogenate combined with an increased amount of CSL to 30 g/l did not perform as well as the combination of less CSL and small amounts of yeast homogenate as shown in row 16. Nonetheless, even with the complete absence of yeast homogenate and the presence of high amounts of CSL alone, the titer, yield and productivity of the strain grown in the presence of betaine was still more than twice that obtained in the absence of betaine with 15 g/l yeast homogenate and more than triple that obtained in the absence of betaine and the absence of yeast homogenate (compare row 16 to rows 1 and 2).

Having determined that betaine could greatly improve the ability of original strain FZ45.860.30 to produce succinate from CSL as the primary supplement in the absence of yeast extract, a derivative, CSL adapted strain designated ME2.7F was tested for the ability to produce succinate in the presence of betaine without any additional yeast homogenate. Strain ME2.7f was derived by successively growing cultures of FZ45.860.30 in liquid media lacking yeast extract and containing 0.5-1.0 g/l betaine, dextrose, CSL and monosodium glutamate, pH adjusted with sodium carbonate, and selecting rapidly growing colonies on agar plates of the same media. The results shown in rows 17-21 demonstrate that a requirement for yeast extract was completely abolished, and that strain ME2.7F could in fact, produce succinate from dextrose with media supplemented with CSL in the presence of betaine, as well or better than the parental strain FZ45.830.60 could produce succinate in the presence of local yeast homogenate or commercial yeast extract in the presence or absence of betaine.

Genetic sequencing of the parent strain and ME2.7f showed that ME2.7F had incorporated, through spontaneous mutations, a change from an isoleucine to lysine at amino acid position 417 for gene encoding a choline/carnatine/betaine transporter located at chromosomal map position 93.9 of the A. succinogenes genome. This change is believed to most significantly influence the ability of betaine to improve conversion of the sugar to succinate. Two other notable mutations less likely to impact the improvements in production specifically due to betaine, but potentially contributing more generally to improved C4 diacid production are: 1) a change from a glycine to a cysteine at amino acid position 59 for a gene encoding transcriptional factor FruR located at map position 252.3, which is a transcriptional factor that binds D fructose and is involved in the regulation of operons for central pathways in carbon metabolism; and less likely, 2) a change from glutamine to lysine at amino add position 498 for a gene encoding a sodium proline symporter protein located at map position 151.3.

TABLE 1

| | | Corn | | | Betaine | | | | | |
|---|---|---|---|---|---|---|---|---|---|---|
| | | Steep | Homogenized | | free base | | | | Rate | Residual |
| | | Liquor | Yeast [g/l] | MSG | anhydrous | Fermentation | Titer | Yield | g ‘1’ | Dextrose |
| | Experiment | [g/l] dsb | dsb | [g/l] | [g/l] | Time [hrs] | [g/l] | [%] | hr | [g/l] |
| 1 | SA0809X1 | 7 | 0 | 0 | 0 | 73.5 | 29 | 20 | 0.4 | 104.0 |
| 2 | SA1018X4 | 3.5 | 15 | 1.3 | 0 | 45 | 41 | 30 | 0.9 | 86.9 |
| 3 | SA1018X1 | 3.5 | 15 | 1.3 | 1 | 36 | 90 | 86 | 2.5 | 0.0 |
| 4 | SA1018X2 | 3.5 | 15 | 1.3 | 1 | 39.5 | 94 | 85 | 2.4 | 0.2 |
| 5 | SA1020X2 | 3.5 | 15 | 1.3 | 1 | 36 | 104 | 85 | 2.9 | 0.0 |
| 6 | SA1021X2 | 3.5 | 15 | 0.2 | 0.5 | 54 | 115 | 94 | 2.2 | 0.4 |
| 7 | SA1021X1 | 3.5 | 15.1 | 1.3 | 1 | 42 | 109 | 92 | 2.7 | 0.2 |
| 8 | SA1023X1 | 3.5 | 15 | 1.3 | 1 | 48 | 102 | 90 | 2.1 | 0.6 |
| 9 | SA1111X1 | 3.5 | 15 | 0.5 | 0 | 67 | 54 | 39 | 0.8 | 68.0 |
| 10 | SA1111X2 | 3.5 | 15 | 0.5 | 0.125 | 67 | 91 | 78 | 1.4 | 2.8 |
| 11 | SA1111X3 | 3.5 | 15 | 0.5 | 0.25 | 67 | 88 | 74 | 1.3 | 5.3 |
| 12 | SA1112X1 | 20 | 5 | 0.5 | 0 | 68 | 56 | 42 | 0.8 | 60.5 |
| 13 | SA1112X2 | 20 | 5 | 0.5 | 0.125 | 68 | 100 | 85 | 1.5 | 5.4 |
| 14 | SA1112X3 | 20 | 5 | 0.5 | 0.5 | 36 | 98 | 87 | 2.7 | 0.0 |
| 15 | SA1112X4 | 20 | 5 | 0.5 | 1 | 42 | 95 | 83 | 2.3 | 0.1 |
| 16 | SA1109X1 | 30 | 0 | 0.5 | 0.5 | 48 | 74 | 45 | 1.5 | 68.9 |
| 17 | SA1108X3- | 30 | 0 | 0.5 | 0.5 | 30 | 83 | 78 | 2.8 | 0.0 |
| | ME2.7F | | | | | | | | | |
| 18 | SA1108X4- | 30 | 3 | 0.5 | 0.5 | 30 | 85 | 73 | 2.8 | 0.0 |
| | ME2.7F | | | | | | | | | |
| 19 | SA1109X2: | 30 | 0 | 0.5 | 0.5 | 43 | 103 | 68 | 2.4 | 0.5 |
| | ME2.7F | | | | | | | | | |
| 20 | SA1109X3: | 30 | 0 | 0.5 | 0.25 | 60 | 104 | 68 | 1.7 | 0.3 |
| | ME2.7F | | | | | | | | | |
| 21 | SA1109X4: | 30 | 0 | 0.5 | 0.5 | 50.5 | 103 | 67 | 2 | 0.4 |
| | ME2.7F | | | | | | | | | |

The amount of betaine needed to provide these results described herein is on the order of 0.1 to 1 g/liter, with substantially equivalent results being obtained with amounts as low as 0.125 g/liter which is slightly above 1 mM. The succinic acid produced, however, is on the order of 70-115 g/liter, which corresponds to a concentration of about 590-980 mM. Accordingly, it is doubtful that the sole effect of the betaine is as an osmoprotectant as is believed to be the case in other bacterial fermentations, for example to make lactic acid from E. coli or lysine from Corynebacteria. The beneficial betaine concentration is also too low to be acting as a methyl donor or other consumable reagent for the production of the product, which is believed to be the case for the production of vitamin B₁₂ from Pseudomonas.

While not being bound, by theory, it is believed that the beneficial effects of betaine in the production of C4 diacids may be through use thereof as is regenerable electron donor for redox reactions in the cell. In this regard, another surprising and unexpected result was the discovery that biotin and other vitamins previously thought to be necessary for high production of C4 diacids from A. succinogenes did not statistically increase the yield from glucose and could be eliminated altogether when betaine was provided as the sole non-nutrative supplement in the fermentation media.

### Example Materials and Methods

Local yeast homogenate was obtained by aerobically growing an ethanol producing strain of the bakers yeast S. cerevesiae on dextrose until the culture reached stationary phase, recovering the yeast cells as paste, and homogenizing the yeast paste with an APV Gaulin French press type homogenizer at a pressures of 12,500 psi or greater. The glutamic acid was added in the form of monosodium glutamate, and the betaine was betaine base anhydrous from MP Biomedicals

Inoculum Medium A. succinogenes strain FZ45830,60, provided by MBI International, was grown in two 100 ml serum bottles each containing 50 ml of inoculum medium incubated at 38° C. for 15-24 hours by shaking in a rotary incubator at 180 rpm, which was then used as 5% vol/vol inoculum for final fermenter volume of 2.0 liters in a 7.5 lite fermenter. The inoculum medium was prepared in two steps. In the first step, 4 g of MgCO₃ was weighed into a 100 ml Wheaton serum bottle containing 30 ml of distilled water, which was then sealed, autoclaved and cooled. In the second step the remaining ingredients, which were prepared as supplemental sterilized solutions, were added to the serum bottle with a sterile syringe. The supplemental solutions and formulation of the inoculum media are shown below.

Vial Media

| | Amount (ml) |
|---|---|
| Solution | per 50 ml |
| Pre-Prepared MgCO₃ (133 g/l) | 30.0 |
| Tastone 900AG yeast extract (10% solution) if added | As targeted |
| Sodium phosphate buffer (0.5M solution: | 0.5 |
| Na₂HPO₄-51 g/l, NaH₂PO₄•H₂O - 19.3 g/l) | |
| Na₂CO₃ (20% solution) | 1.0 |
| Biotin 20 mg/l if added | 0.5 |
| Kanamycin 50 mg/ml | 0.1 |
| Corn Steep Liquor(ADM, Decatur, IL) | As targeted |
| 15.1% g/l dry solids basis (dsb) | |
| Dextrose 64% | 8.0 |
| Distilled Water | To 50 ml total |

The serum bottle was inoculated with 1.5 ml of an overnight culture grown in the same medium. The serum bottle was flushed and overlaid with CO₂ before incubation

Seed Medium A New Brunswick EPPENDORF BiOFLO 310-7.5 liter fermenter was inoculated with the 2 serum bottles containing the inoculation culture for initial production fermentation studies or as a seed culture for larger scale fermentations. The operating conditions are listed below:

| | | |
|---|---|---|
| Agitation | 250 | rpm |
| Gas flow | 0.025 | vvm CO₂ |
| Temperature | 38° | C. |

| | |
|---|---|
| pH | 6.90 |

The seed medium contained 4 g/l CSL dsb, 14 g/l TASTONE 900AG yeast extract, 0.0002 g/l biotin, 10 ml/l 0.5M sodium phosphate buffer and 100 g/l dextrose. The pH was adjusted with a 35% Mg(OH)₂ slurry. The seed fermenter was inoculated at 5% v/v from the incubated serum bottle culture and was harvested in the early log phase (e.g., an increase in OD of 3 to 4 units) other measure of early log phase?) Two hundred ml of the seed culture was used to inoculate 4 liters of media in a second 7.5 liter production fermenter.

Production The SA1018.X1 production media formulation contained as indicated. 3.5 to 15 g/l dry solids basis (dsb) local yeast homogenate 3.5 to 30 g/l dsb corn steep liquor, 0 to 1 g/l betaine free base anhydrous, 0 to 1.3 g/l glutamic acid, 92 to 115 g/l dextrose, 0.0002 g/l biotin, 0.66 g/l Na2HPO4, 0.25 g/l NaH2PO4*H2O, 3.2 g/l Na2CO3 with a 150 g dextrose feed starting at 12 hours into the fermentation (i.e. a solution containing a total of 150 dextrose at 70% wt/vol was fed a constant rate from hours 12 to 22 or 24 of the fermentation run. The 5A1018.X2 production media and conditions were the same as SA1018.X1 but used 92 g/l dextrose and biotin was omitted. The SA1018 X4 production media and conditions were the same as SA1018.X1 but used 104 g/l dextrose, and both biotin and betaine were omitted. All production scale fermentations were continuously pH adjusted with a mixture of Mg(OH)₂ and KOH (4.5 mol/3 mol) to maintain the pH at 6.9.

1. A method of fermenting a microorganism to make a C4 diacid, comprising
growing a microorganism that produces the C4 diacid in a production medium containing a sugar and an effective amount of betaine to yield at least 60% conversion of the sugar to the C4 diacid at the end of a fermentation period defined by when the sugar is exhausted from the medium or ceases to be consumed, wherein growing the microorganism under the same conditions in the same production medium but lacking the betaine produces a yield of greater than 10% but less than 60% conversion of the sugar to the C4 diacid.

2. The method of claim 1 wherein the medium does not contain yeast extract.

3. The method of claim 1 wherein the microorganism is A. succinogenes, and the C4 diacid is succinic acid.

4. The method of claim 3 wherein the medium does not contain yeast extract.

5. The method of claim 3 wherein the medium does not contain added biotin.

6. The method of claim 3 wherein the yield is at least 70% conversion of the sugar to the C4 diacid.

7. The method of claim 3 wherein the yield is at least 80% conversion of the sugar to the C4 diacid.

8. The method of claim 3 wherein the medium contains yeast extract and the yield is at least 90% conversion of the sugar to the C4 diacid.

9. The method of claim 3 wherein a productivity rate for the production of the C4 diacid during the fermentation period is at least 1.5 enter per hour.

10. The method of claim 9 wherein the productivity rate is at least 2.0 enter per hour.

11. The method of claim 9 wherein the productivity rate is at least 2.5 g/liter per hour.

12. The method of claim 3 wherein the medium contains corn steep liquor as an added nutrient source.

13. (canceled)

14. (canceled)

15. The method of claim 1 wherein the effective amount of betaine is between 0.01 and 1.0 g/liter.

16. (canceled)

17. A method for improving a C4 diacid production from a C4 diacid producing microorganism comprising,
obtaining a C4 producing microorganism that can produce at least 60% conversion of a sugar to the C4 diacid at the end of a fermentation period in a fermentation medium containing the sugar and yeast extract but that is not able to covert at least 60% of the sugar to the C4 diacid under the same conditions in a comparative medium that is the same but lacks the yeast extract;
passing a culture of the microorganism between a liquid medium and a platting medium, each being the comparative medium lacking the yeast extract but containing at least 0.1 g/liter betaine, and selecting colonies of the culture that grow most rapidly on the platting medium; and
repeating the passage of the culture between the liquid medium and the platting medium until a colony is selected that can produce at least 60% conversion of a sugar to the C4 diacid at the end of the fermentation period in the comparative medium lacking the yeast extract and including the betaine.

18. The method of claim 17 wherein the microorganism is A. succinogenes, and the C4 diacid is succinic acid.

19. The method of claim 17 wherein the selected colony can produce at least 60% conversion of the sugar to the C4 diacid at the end of the fermentation period in the comparative medium lacking the yeast extract and including the betaine but also lacking added biotin.

20. A strain of A. succinogenes made by the method of claim 18.

21. A strain of C4 diacid producing bacterium that comprises a mutation in a betaine transporter gene that enables the bacterium to produce at least at least 60% conversion of a sugar in a fermentation medium to the C4 diacid at the end of a fermentation period defined by when the sugar is exhausted from the medium or ceases to be consumed, and wherein the medium contains an effective amount of betaine.

22. The strain of claim 21 wherein the bacterium is A. succinogenes.

23. The strain of claim 22 wherein the mutation changes an isoleucine to lysine at amino acid position 417 of the betaine transporter gene.
